(19) United States
(12) Patent Application Publication
(10) Pub. No.: US 2023/0265410 A1
(43) Pub. Date: Aug. 24, 2023

(54) CELL LINES, SYSTEMS, KITS, AND METHODS FOR DETERMINING THE SPECIFICITY AND/OR POTENCY OF CERAMIDASE INHIBITORS

(71) Applicant: UNIVERSITY OF LOUISVILLE RESEARCH FOUNDATION, INC., Louisville, KY (US)

(72) Inventors: Mark A. Doll, Louisville, KY (US); Leah Siskind, Louisville, KY (US)

(21) Appl. No.: 18/162,777

(22) Filed: Feb. 1, 2023

Related U.S. Application Data
(60) Provisional application No. 63/305,457, filed on Feb. 1, 2022.

(51) Int. Cl. C12N 9/80; G01N 33/50

(52) U.S. Cl. CPC C12N 9/80; C12Y 305/01023; G01N 33/5023

(57) ABSTRACT

Described herein are cell lines, systems, kits, and methods for determining the specificity and potency of inhibitors for human ceramidase inside the cell and in a test tube.

### CROSS-REFERENCE TO RELATED APPLICATIONS

This application claims benefit of U.S. Provisional Application No. 63/305,457, filed Feb. 1, 2022, which is hereby incorporated herein by reference in its entirety.

### BACKGROUND

The sphingolipids are a family of membrane lipids derived from the aliphatic amino alcohol sphingosine and its related sphingoid bases. Sphingolipids are present in eukaryote membranes, where they exert important structural roles in the regulation of fluidity and subdomain structure of the lipid bilayer. In addition, they have emerged as key effectors in many aspects of cell biology including inflammation, cell proliferation and migration, senescence and apoptosis.

Ceramide is considered a central molecule in sphingolipid catabolism. The generic term “ceramide” comprises a family of several distinct molecular species deriving from the N-acylation of sphingosine with fatty acids of different chain length, typically from 14 to 26 carbon atoms. Ceramide can be synthesized de novo from condensation of serine with palmitate, catalyzed by serine palmitoyltransferase, to form 3-keto-dihydrosphingosine. In turn, 3-keto-dihydrosphingosine is reduced to dihydrosphingosine, followed by acylation by a (dihydro)-ceramide synthase. Ceramide is formed by the desaturation of dihydroceramide. Alternatively, ceramide can be obtained by hydrolysis of sphingomyelin by sphingomyelinases. Ceramide is metabolized by ceramidases to yield sphingosine and fatty acid.

Ceramide plays an important role in a variety of cellular processes. Ceramide concentrations increase in response to cellular stress, such as DNA damage, exposure to cancer chemotherapeutic agents and ionizing radiation, and increased ceramide levels can trigger senescence and apoptosis in normal cells. Moreover, ceramide is also involved in the regulation of cancer cell growth, differentiation, senescence and apoptosis. Many anticancer drugs increase ceramide levels in cells by stimulating its de novo synthesis and/or hydrolysis of sphingomyelin. For example, daunorubicin elicits ceramide production through the de novo pathway. De novo ceramide induction was observed in various human cancer cells after treatment with camptothecin and fludarabine, and with gemcitabine. In many of these studies, inhibition of de novo ceramide synthesis was found to prevent, at least in part, the cytotoxic responses to these agents, thus indicating that the de novo pathway might function as a common mediator of cell death. Therefore, increasing or sustaining the levels of ceramide in cancer cells could be envisaged as a novel therapeutic strategy to induce cancer cell death.

One approach to increase or sustain the levels of ceramide in cells is to inhibit the enzymes responsible for ceramide clearance. Enzymes that contribute to decreasing the intracellular levels of ceramide are glucosylceramide synthase, which incorporates ceramide into glucosylceramide, sphingomyelin synthase, which synthesizes sphingomyelin, and ceramidases, which hydrolyze ceramide to sphingosine and fatty acid. Currently, there are five known human ceramidases: acid ceramidase (AC), neutral ceramidase, alkaline ceramidase 1, alkaline ceramidase 2, and alkaline ceramidase 3. Among them, acid ceramidase is emerging as an important enzyme in the progression of cancer and in the response to tumor therapy. Messenger RNA and protein levels of acid ceramidase are heightened in a wide variety of cancers including prostate cancer, head and neck cancer, and melanoma. In prostate cancer, acid ceramidase expression correlates with the malignant stage of the disease. Up-regulation of acid ceramidase has also been observed in prostate cancer cells in response to radiotherapy, and this mechanism desensitizes cells to both chemotherapy and radiotherapy. Restoration of acid ceramidase levels in radio-resistant cells by either gene silencing or inhibition of acid ceramidase activity confers radiation sensitivity to prostate cancer cells. Improvement of tumor sensitivity to ionizing radiation by inhibition of acid ceramidase has been shown in vivo in a PPC-1 xenograft model. Together, these data suggest that acid ceramidase provides a growth advantage to cancer cells and contributes to the altered balance between proliferation and death eventually leading to tumor progression. Therefore, inhibition of acid ceramidase appears to be a promising strategy for cancer treatment.

Neutral and alkaline ceramidases have also been examined as a physiological target for a variety of diseases. Neutral ceramidase inhibition has been shown to be protective in rodent models of colon cancer, acute kidney injury, and traumatic brain injury. Alkaline ceramidases play a role in hepatocellular carcinoma and several types of leukemias. Therefore, development of inhibitors for the specific ceramidases may be a promising for the treatment of a variety of diseases.

The aforementioned balance between cellular proliferation and death is mainly regulated by the ceramide/sphingosine 1-phosphate S1P rheostat. Compelling evidences implicate this pathway as contributor to inflammatory conditions and pain of diverse etiologies. Blocking a particular ceramidase implies an upstream inhibition of ceramide to sphingosine 1-phosphate S1P pathway and, therefore, seems to be a promising approach to inflammatory and pain conditions treatment. Since the specific ceramidases are expressed in particular tissues and in subcellular compartments, specific inhibitors are needed for specific inflammatory and pain conditions. In addition, a specific and potent inhibitor of a particular ceramidase that is ineffective against the other ceramidases may avoid the toxicity that would likely result from inhibiting multiple types of ceramidases.

A variety of ceramidase inhibitors have been developed and investigated, including compounds containing a sphingoid base, a derivative of a sphingoid base, or a salt of a sphingoid base, cyclopropenyl-sphingosine derivatives, cationic ceramide derivatives, oleoylethanolamides, quinolinones, and 2,4-dioxopyrimidine-1-carboxamides. However, at present, there is no way to evaluate how specific and potent these inhibitors are for each human ceramidase in situ. The availability of such screening techniques offers the potential to improve development of ceramidase inhibitors.

### SUMMARY

As discussed above, ceramidases are known to be involved in various diseases states, including cancer, Alzheimer's disease, and Farber disease to name a few. While ceramidases have been studies for decades, our knowledge of their role in human disease and their role in human physiology remains limited. Inhibitors of various ceramidase have been developed especially for ASAH1 but there is no way to specifically test how specific and potent these inhibitors are for each human ceramidase.

Described herein are cell lines, systems, kits, and methods for determining the specificity and potency of inhibitors for human ceramidase inside the cell (in situ) and in a test tube. These were accomplished by deleting other ceramidase in a mouse cell line and then overexpressing each human ceramidase separately using a doxycycline-inducible overexpression system. These cell lines were tested against commercially available ceramidase inhibitors to confirm the efficacy of these screening methods. Using these cell lines, the specificity and potency of existing and potential new inhibitors of human ceramidases can be determined both in vitro and most importantly in situ. These cell lines, kits, and methods can be used to evaluate new and existing inhibitors to identify compounds that specifically target individual ceramidases of interest. Such targeted inhibitors have potential applications in a variety of therapeutic methods.

### DESCRIPTION OF DRAWINGS

FIG. 1 is a flow chart summarizing the workflow of experiments described in the Examples.

FIGS. 2A-2B show in vitro and in situ ceramidase assays performed on Asah2^{+/+} (WT) and Asah2^{-/-} (KO) mouse embryonic fibroblast cells (MEFs). FIG. 2A shows in vitro neutral ceramidase (nCDase) activity assays performed on WT and KO MEF cell lysates. Briefly, reactions contained lysate (50 μg protein), 25 μM C12-NBD-ceramide in 50 μL reaction buffer (50 mM HEPES, 150 mM sodium chloride and 1% sodium chloate, pH 7.2). The in vitro reactions were initiated upon addition of cellular lysate and were incubated at 37° C. for 0 to 4 h. The reaction was stopped with the addition of 150 μL of chloroform/methanol (50:50, v/v). The organic phase of the extraction was removed, evaporated to dryness, and resuspended in 5 μL of chloroform:methanol (50:50 v/v) and spotted on TLC plate, which was run as directed in methods to separate and measure fluorescent metabolites. FIG. 2B shows in situ ceramidase assays performed on WT and KO MEFs grown in culture. Briefly 25,000 cells for both WT and KO MEFs were plated in separate wells of a 96-well plate and allowed to attach overnight. The next morning, the media was removed and replaced with media containing 25 μM C12-NBD-ceramide. Cells were allowed to grow and metabolize the C12-NBD-ceramide for the indicated time periods. Media containing metabolized C12-NBD-ceramide was then collected, extracted, evaporated to dryness, loading and separation of substrate and product by TLC.

FIGS. 3A-3B show the estimation of different ceramidase mRNA levels in WT and KO ASAH2 MEFs and the screening of clones with Asah1 and Acer3 knocked out in KO Asah2 MEFs. As shown in FIG. 3A, RNA was isolated from WT and KO MEFs grown in culture. RNA was converted to cDNA and used for running real-time RT-PCR to compare relative expression of the mouse ceramidases in the WT and KO MEFs. The CT numbers are Cycle Thresholds with lower number indicating higher expression of the gene of interest. Acer 3 and Asah1 mRNA are expressed at high levels suggesting that Acer 3 and Asah1 are the enzymes interfering with us measuring Asah2 activity in situ. As shown in FIG. 3B, KO MEFs cells stably expressing CAS9 protein were transfected with gRNA's targeting Acer3 and Asah1. Forty-eight hours after transfection cells were plated at low density to allow individual cells to grow up and form individual clones, which were isolated and allowed to grow up. Each clone was analyzed for in situ ceramidase activity as was described in methods. Briefly, 25,000 cells of each clone were separately plated into wells of a 96-well plate and allowed to attach overnight. The next morning, media was removed and replaced with media containing 25 μM C12-NBD-ceramide. Cells were allowed to grow and metabolize the C12-NBD-ceramide for 24 h. Media containing metabolized C12-NBD-ceramide was collected, extracted and load as described in the methods section. Lanes 1 thru lane 11 are each different clones of KO MEFs transfected with gRNA's against ASAH1 and ACER3. Lane 12=in vitro assay using purified recombinant human ASAH2.

FIGS. 4A-4B show in vitro (FIG. 4A) and in situ (FIG. 4B) ceramidase assays of Human ASAH2 overexpression clones and other cells lines. Parental WT and KO Asah2 MEFs, KO Asah2 MEFs with Acer3 and Asah1 knocked out (KOAA10 or 12) and Ko Asah2 MEFs with Acer3 and Asah1 knocked out plus dox-inducible ASAH2 gene (KoAA10 or 12+ASAH2) were grown +/−doxycycline for 24 h prior. Doxycycline induced cells were harvested and cell lysates were prepared for in vitro assays. The in vitro reactions contained cell lysate (50 μg), 25 μM C12-NBD-ceramide and reaction buffer (50 mM HEPES, 150 mM sodium chloride and 1% sodium chloate, pH 7.2. In vitro reactions (50 μl) were run for 4 h and stopped by adding chloroform/methanol. Fluorescent metabolites were extracted and separated by TLC as described in methods. For in situ cells (25,000) were plate in normal media+/− doxycycline (1 ug/ml) and incubated overnight. The next morning, media was replaced with media containing c12-NBD-ceramide (+/−doxycycline) and were incubated with for 24 h before collecting the media and extracted before being loaded and separated by TLC as described in methods.

FIGS. 5A-5C provide a comparison of mRNA expression and protein activity (in vitro and in situ) of human ceramidases in overexpression clones. As shown in FIG. 5A, RNA was isolated and cDNA made from human ceramidase overexpression clones. Real-time PCR was used to measure and compare relative expression of each ceramidase. Individual clone with highest mRNA expression of each ceramidase were chosen for further studies. FIG. 5B shows the protein activity of selected human ceramidases overexpression clones measured using in vitro assays. In vitro reactions were initiated upon addition of equal amounts of cellular lysate from cells induced with doxycyline and were incubated at 37 C for 0 to 4 h. The reaction was stopped with the addition of 150 μL of chloroform/methanol and extraction and evaporated to dryness and run on the TLC as directed in methods. FIG. 5C shows the e protein activity of selected human ceramidases overexpression clones measured using in situ assays. Cell lines (25,000) were plated in a 96-well plate and grown overnight in the presence of +/−doxycycline (1 ug/ml). The next morning media was removed and replaced with media containing 25 μM C12-NBD-ceramide. Cells were allowed to grow and metabolize the C12-NBD-ceramide for 24 h. Media containing metabolized C12-NBD-ceramide was collected, extracted, and loaded.

FIGS. 6A-6B show the specificity and potency of commercially available ceramidase inhibitors determined both in vitro (FIG. 6A) and in situ (in cell, FIG. 6B) using the using the new human ceramidase overexpression clones. Briefly for in vitro reactions, cell lysates of overexpressed human ceramidase were prepared from cell lines incubated with doxycycline (1 ug/ml) overnight before harvesting and lysing the cells. In vitro reactions were performed by incubating the cell lysate for 1 h with the various inhibitors (37° C.) and then after the 1 h incubation 25 μM RBM14c12 (ASAH1 and ASAH2) or RBM14c16 (ACER2 and ACER3) were added and incubated at 37° C. for additional 4 h before stopping the reaction and detection. In situ reactions were performed by plating 25,000 cell/well in a 96-well plate and incubating them in the presence of doxycycline (1 μg/ml) overnight. The next morning, media was replaced with media containing inhibitor for 1 h before adding RBM14c12 (ASAH1 and ASAH2) or RBM14c16 (ACER2 and ACER3). Cells were then allowed to metabolize substrate for 4 h before removing the media to a new plate and adding 25 μL of methanol to stop the reaction. One-hundred microliters of 2.5 mg/mL sodium periodate in 0.1 M Glycine/NaOH pH 10.6 was added and incubated for 1 h and before reading fluoresecence on the plate reader (excitation/emission 355/460). Alamar Blue post incubation was used to measure the toxicity caused by incubation with the inhibitor.

### DETAILED DESCRIPTION

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

At various places in the present specification, divalent linking substituents are described. Where the structure clearly requires a linking group, the Markush variables listed for that group are understood to be linking groups.

As detailed above, ceramidases are known to be involved in various diseases states, including cancer, Alzheimer's disease, and Farber disease. While ceramidases have been studied for decades, knowledge of their role in human disease and their role in human physiology remains limited. Inhibitors of various ceramidase have been developed, especially for ASAH1. However, there is no established method for assaying how specific and potent these inhibitors are for each human ceramidase in situ.

Described herein are cell lines, systems, kits, and methods for determining the specificity and potency of inhibitors for human ceramidase inside the cell and in a test tube.

Cells and Cell Lines

Provided herein are modified non-human cells deficient in the expression of one or more endogenous ceramidase genes (e.g., ASAH1, ASAH2, ASAH2B, ASAH2C, ACER1, ACER2, ACER3, or any combination thereof) and comprising one or more nucleic acids encoding a human ceramidase. In some embodiments, the one or more endogenous ceramidase genes are selected from ASAH1, ASAH2, ACER1, ACER2, ACER3, or any combination thereof. In certain embodiments, the cells are deficient in the expression of ASAH1, ASAH2, ACER1, ACER2, and ACER3. In certain embodiments, the cells deficient in the expression of all endogenous ceramidase genes. In certain embodiments, the cell exhibits no detectable ceramidase activity prior to introduction of the one or more nucleic acids encoding a human ceramidase.

The one or more nucleic acids encode a human ceramidase can be selected from the group consisting of ASAH1, ASAH2, ASAH2B, ASAH2C, ACER1, ACER2, ACER3, or any combination thereof. In some embodiments, the one or more nucleic acids encode a human ceramidase are selected from the group consisting of ASAH1, ASAH2, ACER1, ACER2, ACER3, or any combination thereof. In certain embodiments, the one or more nucleic acids encode a single human ceramidase (e.g., a human ceramidase selected from the group consisting of ASAH1, ASAH2, ACER1, ACER2, ACER3, or any combination thereof).

In some embodiments, the modified non-human cell is deficient in the expression of one or more endogenous ceramidase genes (ASAH1, ASAH2, ACER1, ACER2, ACER3, or any combination thereof) and comprises a nucleic acid encoding human ASAH1. In some embodiments, the modified non-human cell is deficient in the expression of endogenous ceramidase genes ASAH1, ASAH2, ACER1, ACER2, and ACER3, and comprises a nucleic acid encoding human ASAH1. In some embodiments, the cell is deficient in the expression of all endogenous ceramidase genes and comprises a nucleic acid encoding human ASAH1. In certain embodiments described in this paragraph, ASAH1 is the single human ceramidase encoded for by nucleic acids present in the cell.

In some embodiments, the modified non-human cell is deficient in the expression of one or more endogenous ceramidase genes (ASAH1, ASAH2, ACER1, ACER2, ACER3, or any combination thereof) and comprises a nucleic acid encoding human ASAH2. In some embodiments, the modified non-human cell is deficient in the expression of endogenous ceramidase genes ASAH1, ASAH2, ACER1, ACER2, and ACER3, and comprises a nucleic acid encoding human ASAH2. In some embodiments, the cell is deficient in the expression of all endogenous ceramidase genes and comprises a nucleic acid encoding human ASAH2. In certain embodiments described in this paragraph, ASAH2 is the single human ceramidase encoded for by nucleic acids present in the cell.

In some embodiments, the modified non-human cell is deficient in the expression of one or more endogenous ceramidase genes (ASAH1, ASAH2, ACER1, ACER2, ACER3, or any combination thereof) and comprises a nucleic acid encoding human ACER1. In some embodiments, the modified non-human cell is deficient in the expression of endogenous ceramidase genes ASAH1, ASAH2, ACER1, ACER2, and ACER3, and comprises a nucleic acid encoding human ACER1. In some embodiments, the cell is deficient in the expression of all endogenous ceramidase genes and comprises a nucleic acid encoding human ACER1. In certain embodiments described in this paragraph, ACER1 is the single human ceramidase encoded for by nucleic acids present in the cell.

In some embodiments, the modified non-human cell is deficient in the expression of one or more endogenous ceramidase genes (ASAH1, ASAH2, ACER1, ACER2, ACER3, or any combination thereof) and comprises a nucleic acid encoding human ACER2. In some embodiments, the modified non-human cell is deficient in the expression of endogenous ceramidase genes ASAH1, ASAH2, ACER1, ACER2, and ACER3, and comprises a nucleic acid encoding human ACER2. In some embodiments, the cell is deficient in the expression of all endogenous ceramidase genes and comprises a nucleic acid encoding human ACER2. In certain embodiments described in this paragraph, ACER2 is the single human ceramidase encoded for by nucleic acids present in the cell.

In some embodiments, the modified non-human cell is deficient in the expression of one or more endogenous ceramidase genes (ASAH1, ASAH2, ACER1, ACER2, ACER3, or any combination thereof) and comprises a nucleic acid encoding human ACER3. In some embodiments, the modified non-human cell is deficient in the expression of endogenous ceramidase genes ASAH1, ASAH2, ACER1, ACER2, and ACER3, and comprises a nucleic acid encoding human ACER3. In some embodiments, the cell is deficient in the expression of all endogenous ceramidase genes and comprises a nucleic acid encoding human ACER3. In certain embodiments described in this paragraph, ACER3 is the single human ceramidase encoded for by nucleic acids present in the cell.

The cells described above can be any suitable non-human cells. In some embodiments, the cells comprise animal cells (e.g., mammalian cells). In some examples, the cell can comprise mouse, rat, pig, bovine, primate, canine, or feline cells. In certain embodiments, the cell can comprise a mouse cell.

The reduced expression of the one or more endogenous ceramidase genes in the cells described above can be obtained by any suitable method known in the art, such as by deletion of all or part of the ceramidase gene, siRNA or oligonucleotide targeting the endogenous ceramidase gene, an inducible knockout system, or CRSIPR-CAS9.

Systems and Kits

Also provided are system for detecting the specificity and/or potency of a ceramidase inhibitor comprising the modified non-human cells described above.

In some embodiments, the system can include a plurality of different modified cells specifically expressing individual human ceramidases of interest. By way of example, in some embodiments the system can comprise a first modified non-human cell, a second modified non-human cell, a third modified non-human cell, a fourth modified non-human cell, and a fifth modified non-human cell. The first modified non-human cell can be deficient in the expression of one or more endogenous ceramidase genes and comprises a nucleic acid encoding human ASAH1. The second modified non-human cell can be deficient in the expression of one or more endogenous ceramidase genes and comprises a nucleic acid encoding human ASAH2. The third modified non-human cell can be deficient in the expression of one or more endogenous ceramidase genes and comprises a nucleic acid encoding human ACER1. The fourth modified non-human cell can be deficient in the expression of one or more endogenous ceramidase genes and comprises a nucleic acid encoding human ACER2. The fifth modified non-human cell can be deficient in the expression of one or more endogenous ceramidase genes and comprises a nucleic acid encoding human ACER3. As discussed above, the one or more endogenous ceramidase genes can be selected from ASAH1, ASAH2, ASAH2B, ASAH2C, ACER1, ACER2, ACER3, or any combination thereof.

In some embodiments, the first modified non-human cell can be deficient in the expression of ASAH1, ASAH2, ACER1, ACER2, and ACER3, and comprises a nucleic acid encoding human ASAH1. In some embodiments, the second modified non-human cell can be deficient in the expression of ASAH1, ASAH2, ACER1, ACER2, and ACER3, and comprises a nucleic acid encoding human ASAH2. In some embodiments, the third modified non-human cell can be deficient in the expression of ASAH1, ASAH2, ACER1, ACER2, and ACER3, and comprises a nucleic acid encoding human ACER1. In some embodiments, the fourth modified non-human cell can be deficient in the expression of ASAH1, ASAH2, ACER1, ACER2, and ACER3, and comprises a nucleic acid encoding human ACER2. In some embodiments, the fifth modified non-human cell can be deficient in the expression of ASAH1, ASAH2, ACER1, ACER2, and ACER3, and comprises a nucleic acid encoding human ACER3.

In some embodiments, the first modified non-human cell can be deficient in the expression of all endogenous ceramidase genes and comprises a nucleic acid encoding human ASAH1. In some embodiments, the second modified non-human cell can be deficient in the expression of all endogenous ceramidase genes and comprises a nucleic acid encoding human ASAH2. In some embodiments, the third modified non-human cell can be deficient in the expression of all endogenous ceramidase genes and comprises a nucleic acid encoding human ACER1. In some embodiments, the fourth modified non-human cell can be deficient in the expression of all endogenous ceramidase genes and comprises a nucleic acid encoding human ACER2. In some embodiments, the fifth modified non-human cell can be deficient in the expression of all endogenous ceramidase genes and comprises a nucleic acid encoding human ACER3.

Also provided are kits for detecting the specificity and/or potency of a ceramidate inhibitor comprising the modified non-human cells described above or the systems described above. Optionally, the kit can further include additional reagents suitable for performing the in vitro and/or in situ (cell based) assays described herein.

Methods and Assays

Also provided herein are methods of detecting the specificity and/or potency of a ceramidase inhibitor. These methods can comprise comprising contacting each modified non-human cell of the system or kit described herein with the ceramidase inhibitor and assaying the expression of the human ceramidase of each cell. Reduced expression of a human ceramidase relative to a non-treated control indicates that the ceramidase inhibitor is specific for the ceramidase expressed in the cell, indicates the potency of the ceramidase inhibitor, or a combination thereof.

Also provided are methods of detecting the specificity of a ceramidase inhibitor comprising obtaining a modified non-human cell deficient in the expression of one or more endogenous ceramidase genes (e.g., ASAH1, ASAH2, ACER1, ACER2, ACER3, or any combination thereof) and comprising one or more nucleic acids encoding a human ceramidase; contacting said modified non-human cell with the ceramidase inhibitor; and assaying the expression of the human ceramidase of each cell. The reduced expression of a human ceramidase relative to a non-treated control indicates that the ceramidase inhibitor is specific for the ceramidase expressed in the cell, indicates the potency of the ceramidase inhibitor, or a combination thereof.

In some embodiments, the one or more endogenous ceramidase genes are selected from ASAH1, ASAH2, ACER1, ACER2, ACER3, or any combination thereof. In certain embodiments, the cells are deficient in the expression of ASAH1, ASAH2, ACER1, ACER2, and ACER3. In certain embodiments, the cells deficient in the expression of all endogenous ceramidase genes. In certain embodiments, the cell exhibits no detectable ceramidase activity prior to introduction of the one or more nucleic acids encoding a human ceramidase.

The one or more nucleic acids encode a human ceramidase can be selected from the group consisting of ASAH1, ASAH2, ASAH2B, ASAH2C, ACER1, ACER2, ACER3, or any combination thereof. In some embodiments, the one or more nucleic acids encode a human ceramidase are selected from the group consisting of ASAH1, ASAH2, ACER1, ACER2, ACER3, or any combination thereof. In certain embodiments, the one or more nucleic acids encode a single human ceramidase (e.g., a human ceramidase selected from the group consisting of ASAH1, ASAH2, ACER1, ACER2, ACER3, or any combination thereof).

In some embodiments, the modified non-human cell is deficient in the expression of one or more endogenous ceramidase genes (ASAH1, ASAH2, ACER1, ACER2, ACER3, or any combination thereof) and comprises a nucleic acid encoding human ASAH1. In some embodiments, the modified non-human cell is deficient in the expression of endogenous ceramidase genes ASAH1, ASAH2, ACER1, ACER2, and ACER3, and comprises a nucleic acid encoding human ASAH1. In some embodiments, the cell is deficient in the expression of all endogenous ceramidase genes and comprises a nucleic acid encoding human ASAH1. In certain embodiments described in this paragraph, ASAH1 is the single human ceramidase encoded for by nucleic acids present in the cell.

In some embodiments, the modified non-human cell is deficient in the expression of one or more endogenous ceramidase genes (ASAH1, ASAH2, ACER1, ACER2, ACER3, or any combination thereof) and comprises a nucleic acid encoding human ASAH2. In some embodiments, the modified non-human cell is deficient in the expression of endogenous ceramidase genes ASAH1, ASAH2, ACER1, ACER2, and ACER3, and comprises a nucleic acid encoding human ASAH2. In some embodiments, the cell is deficient in the expression of all endogenous ceramidase genes and comprises a nucleic acid encoding human ASAH2. In certain embodiments described in this paragraph, ASAH2 is the single human ceramidase encoded for by nucleic acids present in the cell.

In some embodiments, the modified non-human cell is deficient in the expression of one or more endogenous ceramidase genes (ASAH1, ASAH2, ACER1, ACER2, ACER3, or any combination thereof) and comprises a nucleic acid encoding human ACER1. In some embodiments, the modified non-human cell is deficient in the expression of endogenous ceramidase genes ASAH1, ASAH2, ACER1, ACER2, and ACER3, and comprises a nucleic acid encoding human ACER1. In some embodiments, the cell is deficient in the expression of all endogenous ceramidase genes and comprises a nucleic acid encoding human ACER1. In certain embodiments described in this paragraph, ACER1 is the single human ceramidase encoded for by nucleic acids present in the cell.

In some embodiments, the modified non-human cell is deficient in the expression of one or more endogenous ceramidase genes (ASAH1, ASAH2, ACER1, ACER2, ACER3, or any combination thereof) and comprises a nucleic acid encoding human ACER2. In some embodiments, the modified non-human cell is deficient in the expression of endogenous ceramidase genes ASAH1, ASAH2, ACER1, ACER2, and ACER3, and comprises a nucleic acid encoding human ACER2. In some embodiments, the cell is deficient in the expression of all endogenous ceramidase genes and comprises a nucleic acid encoding human ACER2. In certain embodiments described in this paragraph, ACER2 is the single human ceramidase encoded for by nucleic acids present in the cell.

In some embodiments, the modified non-human cell is deficient in the expression of one or more endogenous ceramidase genes (ASAH1, ASAH2, ACER1, ACER2, ACER3, or any combination thereof) and comprises a nucleic acid encoding human ACER3. In some embodiments, the modified non-human cell is deficient in the expression of endogenous ceramidase genes ASAH1, ASAH2, ACER1, ACER2, and ACER3, and comprises a nucleic acid encoding human ACER3. In some embodiments, the cell is deficient in the expression of all endogenous ceramidase genes and comprises a nucleic acid encoding human ACER3. In certain embodiments described in this paragraph, ACER3 is the single human ceramidase encoded for by nucleic acids present in the cell.

In some embodiments, the method can comprise screening the ceramidase inhibitor against one or more human ceramidases individually to assess their activity against that particular ceramidase. By way of example, in some embodiments the method can comprise practicing the method described above with a first modified non-human cell, a second modified non-human cell, a third modified non-human cell, a fourth modified non-human cell, and a fifth modified non-human cell. The first modified non-human cell can be deficient in the expression of one or more endogenous ceramidase genes and comprises a nucleic acid encoding human ASAH1. The second modified non-human cell can be deficient in the expression of one or more endogenous ceramidase genes and comprises a nucleic acid encoding human ASAH2. The third modified non-human cell can be deficient in the expression of one or more endogenous ceramidase genes and comprises a nucleic acid encoding human ACER1. The fourth modified non-human cell can be deficient in the expression of one or more endogenous ceramidase genes and comprises a nucleic acid encoding human ACER2. The fifth modified non-human cell can be deficient in the expression of one or more endogenous ceramidase genes and comprises a nucleic acid encoding human ACER3. As discussed above, the one or more endogenous ceramidase genes can be selected from ASAH1, ASAH2, ASAH2B, ASAH2C, ACER1, ACER2, ACER3, or any combination thereof.

In some embodiments, the first modified non-human cell can be deficient in the expression of ASAH1, ASAH2, ACER1, ACER2, and ACER3, and comprises a nucleic acid encoding human ASAH1. In some embodiments, the second modified non-human cell can be deficient in the expression of ASAH1, ASAH2, ACER1, ACER2, and ACER3, and comprises a nucleic acid encoding human ASAH2. In some embodiments, the third modified non-human cell can be deficient in the expression of ASAH1, ASAH2, ACER1, ACER2, and ACER3, and comprises a nucleic acid encoding human ACER1. In some embodiments, the fourth modified non-human cell can be deficient in the expression of ASAH1, ASAH2, ACER1, ACER2, and ACER3, and comprises a nucleic acid encoding human ACER2. In some embodiments, the fifth modified non-human cell can be deficient in the expression of ASAH1, ASAH2, ACER1, ACER2, and ACER3, and comprises a nucleic acid encoding human ACER3.

In some embodiments, the first modified non-human cell can be deficient in the expression of all endogenous ceramidase genes and comprises a nucleic acid encoding human ASAH1. In some embodiments, the second modified non-human cell can be deficient in the expression of all endogenous ceramidase genes and comprises a nucleic acid encoding human ASAH2. In some embodiments, the third modified non-human cell can be deficient in the expression of all endogenous ceramidase genes and comprises a nucleic acid encoding human ACER1. In some embodiments, the fourth modified non-human cell can be deficient in the expression of all endogenous ceramidase genes and comprises a nucleic acid encoding human ACER2. In some embodiments, the fifth modified non-human cell can be deficient in the expression of all endogenous ceramidase genes and comprises a nucleic acid encoding human ACER3.

These methods can be used as assays to screen potential compounds to identify ceramidase inhibitors of interest (e.g., as possible therapeutics and/or leads for pharmaceutical development). These methods can also be used as assays to identify the particular ceramidase (or ceramidases) inhibited by a known ceramidase inhibitor. These methods can also be used as assays to determine the potency (e.g., the IC50) of a ceramidase inhibitor for a particular ceramidase (or ceramidases).

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

Introduction

Sphingolipids are a class of lipids defined by their 18-carbon amino-alcohol backbones which are made in the ER from non-sphingolipid precursors. Small changes in the basic structure gives rise to the many different types of sphingolipids that play important roles in cell biology and provide many metabolites that regulate cell function. Sphingolipid metabolism can be summed up as an array of interconnected networks that diverge from a single common entry point and come together into a single common breakdown point. Ceramide is at the central hub of sphingolipid metabolism and is synthesized de novo from serine and palmitoyl-CoA. Ceramidases are responsible for hydrolyzing ceramide into sphingosine and free fatty acids. There are three types of ceramidases, acid, neutral, and alkaline, based on their pH required for optimal activity. Acid ceramidase (aCDase) is located within lysosomes, and a genetic deficiency of aCDase causes the lysosomal lipid storage disorder known as Farber's disease. aCDase overexpression has been identified in low-survival-rate colorectal adenocarcinoma and glioblastoma, it has also been observed in node-negative melanoma and breast cancer. Alkaline ceramidases (ACERs) 1, 2, and 3 are primarily located in the endoplasmic reticulum (ER) and Golgi complexes. ACER1 inhibition leads to abnormal hair, alopecia, hyperproliferation, inflammation, an abnormal differentiation of the epidermis, sebaceous gland abnormalities, and infundibulum expansion, as well as an increased trans-epidermal water loss and hypermetabolism with an associated reduction in fat content during aging. ACER2 is upregulated by the tumor suppressor gene p53. Inhibition of ACER3 in cancer cell reduces growth and increases cancer cell apoptosis. Neutral ceramidase (nCDase), which is encoded by N-acylsphingosine amidohydrolase 2 (ASAH2), has been identified not only from human and mammalian organisms but also from certain bacteria. Elevated gene expression of nCDase has been identified in both the plasma membrane and Golgi apparatus of colorectal cancer cells. Thus, ceramidases have been studies for decades, our knowledge of their role in many diseases and role in human physiology remains still limited.

Inhibitors of various ceramidase have been developed, especially for ASAH1 but there is no way to specifically test how specific these inhibitors are for the different human ceramidases. In this example, we demonstrate a method for evaluating ceramidase inhibitors by deleting other ceramidase in a mouse cell line and then overexpressing each human ceramidase separately using a doxycycline inducible overexpression system.

Materials and Methods

Cell lines. Mouse ASAH2 WT and KO embryonic fibroblasts were generated using known procedures. Briefly, mouse embryonic fibroblasts (MEFs) were generated from either wild-type (WT) or ASAH2 [mouse neutral ceramidase (nCDase)]-null C57BL/6 mice that were littermates. Cells were immortalized with dominant-negative p53. MEFs were maintained in Dulbecco's modified Eagle's medium (DMEM) containing 10% FBS and supplemented with L-glutamine, penicillin and streptomycin. All cells were incubated at 37° C. in 5% CO2. Cells were not cultured for more than 30 doublings and were routinely assessed for mycoplasma.

Construction of various cell lines. Mouse Asah2 KO Acer 3/Asah1 KO MEFs cells were created by first infecting Mouse Asah2 KO MEFs cells with virus expressing CAS9 protein using LentiCAS9-blast plasmid (Addgene, Watertown, Mass.) according to manufactures instruction. Forty-eight hours after infection cells that were infected were selected using blasticidin to kill cells not expressing blasticidin resistance gene and CAS9 protein. Forty-eight hours after selections with blasticidin resistant cells were transfected with gRNA's targeting mouse Asah1 (SEQ. ID. 1: g*g*g*gccaaagucuucucacc) and Acer3 (SEQ. ID 2: g*g*g*ccccacgaccuccacau) using Dharmafect 1 according to manufactures instructions. Forty-eight hours after transfection cells were plated at low density to form isolated colonies. Cells were allowed grow until individual cells grew into colonies reaching approximately 50 cells before isolating clones using cloning cylinders and transferring isolated clones to larger cell culture dishes to grow up. ASAH1 and ACER3 double KO clones were screened by in situ ceramidase assay using C12-NBD-Ceramide (as described below) to determine clones that had the absence of any ceramidase activity in situ. Clones screened from in situ ceramidase assay that had no detectable ceramidase activity were sequenced. The sequence of mouse Asah1 and Acer3 checked to verity the change in DNA sequence that resulted in non-fuctional gene products from the actions of gRNA's and CAS9 protein. Once we verified that we had successful KO of Asah1 and Acer3 in clones Asah2 KO MEFs KO Asah1 and Acer3-clones 10 and -12 (KOAA10 and KOAA12) we then moved on to over expression of human ASAH1, ASAH2, ACER2 and ACER3 in KOAA10 cell line.

The coding region of human ceramidases were cloned into the lentiviral vector containing doxycycline (dox) inducible expression cassette pCW57.1 (Addgene) at the SalI/AgeI restriction site using PCR cloning. Human ceramidases were amplified using the following primers: forward primer 5′-atcatcgtcgacgccaccatggattacaaggatgacgacgataagATGCCGGGCCGGAGT (SEQ. ID 3; ASAH1), 5′-atcatcgtcgacgccaccatggattacaaggatgacgataagatggccaaacgcaccttc-3′ (SEQ. ID 4; ASAH2), 5′-atcatcgtcgacgccaccatggattacaaggatgacgacgataagATGGGCGCCCCGCACTGGT-3′ (SEQ. ID 5; ACER2), 5′-atcatcgtcgacgccaccatggattacaaggatgacgacgataagATGGCTCCGGCCGCGGA-3′ (SEQ. ID 6; ACER3) containing Sal1 restriction site for cloning, Kozac sequence for protein translation initiation and a sequence coding for a flag tag. Reverse primer 5′-atcatcaccggtTCACCAACCTATACAAGGGTCAGG-3′(SEQ. ID 7; ASAH1), 5′-atcatcaccggtatctatcaacttttcactaaatagttacaacttc-3′ (SEQ. ID 8; ASAH2), 5′-atcatcaccggtTCACGTGATCTTGACTGATGATTTCT-3′ (SEQ. ID 9; ACER2), 5′-atcatcaccggtTCAATGCTTCCTGAGAGGCTCA-3′ (SEQ. ID 10; ACER3), contained a AgeI restriction site for cloning. PCR reaction set-up was performed on ice, and amplification was done using Phusion polymerase (New England Biolabs (NEB) (Ipswich, Mass.) following manufactures instructions. Amplified PCR product was agarose gel purified using Qiagen QIAEX II Gel Extraction kit (Hilden, Germany) before restriction digest with AgeI and SalI followed by purification with Qiagen PCR purification kit (Qiagen) to remove digest ends. Cut purified PCR product was ligated in similarly cut and gel purified pCW57.1 using T4-ligase (NEB) following manufactures instruction. Ligated vector was transformed in NEB-stable E. coli (NEB) and plated on LB-ampicillin plates following manufacturers instructions. Bacterial colonies were screened by PCR using PCR primers above and positive colonies were grown up to isolated plasmid DNA and sequence each Human ceramidase gene. Sequence verified plasmids were used to make lentivirus in 293T cells (ATCC, Manassas, Va.) according to manufactures instruction (Addgene). Lentivirus particles were used to infect KOAA10 MEFs according to manufactures protocol (Addgene). Infected cells were selected using puromycin (1 ug/ml) for 48 h before plating cells at low density for isolation of individual clones in the presence of puromycin (0.5 ug/ml). Isolated clones were isolated using cloning cylinders and grown up to verify and test for level of overexpression of human ceramidase by treating cells with 1 ug/ml of dox for 24 h prior to measuring ceramidase activity in situ using the C12-NBD-ceramide in situ ceramidase assay listed above. Clones showing the highest over expression of each ceramidase were selected for further studies.

In vitro Neutral ceramidase assay. Cell lysate was prepared from cells grown in culture following dissociation of cells from cell culture dish using 0.25% trypsin. For cell lines overexpressing one of the human ceramidases, cells were grown in 1 ug/ml Doxycycline for 24 h prior to harvesting as above and the cells were washed and lysed as below. Cells were resuspended in cell culture media and spun down at 400×g for 4 min. Media was removed and cells washed once with 1×PBS before lysing cells in 50 ul of 50 mM HEPES, 150 mM sodium chloride and 1% sodium chloate, pH 7.2 (ASAH2), 50 mM sodium acetate pH 4.0 (ASAH1) and 100 mM HEPES, 2 mM CaCL₂ pH 9.0 (ACER2 and ACER3) plus proteinase inhibitors.

The protein concentration of samples was determined using a BCA assay with BSA standards, 50 μg of cell lysate protein, and 25 uM C12-NBD-ceramide (Cayman Chemical, Ann Arbor, Mich.) or RBM14C12 (for ASAH1 and ASAH2) or RBM14c16 (for ACER2 and ACER3) (Avanti Polar Lipids, Alabaster, Ala.).

For C12-NBD-labeled-Ceramide, in vitro reactions (50 uL) were run for 0 to 24 h and stopped by adding 150 uL of 50/50 chloroform/methanol. The stopped reactions were then centrifuged at 15,000×g for 1 min to form two separate layers. The lower organic phase containing fluorescent metabolites was then removed and evaporated to dryness. The pellet was resuspended in 5 μL of 50/50 chloroform:methanol and spotted and separated using thin layer chromatography on a SiliaPlate TLCG (cat #TLG-R10014BK-323) (Silicyle, Quebec Canada) using 90/30/0.5 solution of chloroform/methanol/14.8 N ammonium hydroxide.

In vitro reactions (50 μL) using RBM14C12 or RBM14c16 (25 uM) as substrate were run for 4 h and stopped with 25 μL methanol. After stopping the reaction, 100 μL of 2.5 mg/mL sodium periodate in 0.1 M Glycine/NaOH pH 10.6 buffer was added and incubated in the dark for 1 h before measuring fluorescence at excitation of 355 nm and emission at 460 nm.

Real-time PCR quantitation of mRNA levels of various ceramidase. Mouse ASAH2 WT and KO MEFs were grown as above and allowed to grow for 48 h, before collecting the cells for RNA isolation. RNA was isolated using EZNA Total RNA I (Omega Bio-tek, Norcross, Ga. USA) following manufactures instruction. CDNA was made using 1 ug of total RNA using High-capacity cDNA reverse transcriptase kit (Applied Biosystem, Foster City, Calif.) following manufactures instructions. Real-time PCR was performed using ITAQ Universal Sybr Green master mix and 0.5 uM forward and reverse primers and 20 ng of cDNA follow manufacture instruction. Primers were design using Primer Express Version 3 and made to specifically amplify cDNA as opposed to genomic DNA by designing forward and reverse primers into adjacent exons separated by an intron as large as possible. Primer sequences are as follows: mouse ASAH1 for 5′gcgcggactccgctcta-3′ (SEQ. ID 11) and rev 5′-ctcggtccatacagcagcaaa-3′(SEQ. ID 12), ASAH2 for 5′-gtgtcagatatcaatttgatgggctat-3 (SEQ. ID 13) rev 5′-ccacgctcacaaatgccat-3′ (SEQ. ID 14) ACER1 for 5′-attcacttctactacctgcacagcat-3′(SEQ. ID 15) rev 5′tctggcatctcatactttgcatc-3′ (SEQ. ID 16) ACER2 for 5′-gcgaccaagccttctgtga-3′ (SEQ. ID 17) rev 5′-acgaagcaaggcagatgagaa-3′ (SEQ. ID 18) ACER3 for 5′-ctttgggtaagggaggaggaa-3′ (SEQ. ID 19) rev 5-ctcacgctctcatccgaggt-3 (SEQ. ID 20) B2m predesigned Mm00437762_m1 (Applied Biosystems, Foster City, Calif.).

In situ Ceramidase assay using C12-NBD-Ceramide. Cells (25,000/well) were plated in 96-well plates and grown overnight in the presence of +/−doxycycline (dox) (μg/ml) for 24 h to turn on expression of dox inducible ceramidase. The next morning media was removed and replaced with media containing 25 uM C12-NBD-ceramide+/−dox (μg/ml) and cells allow to grow and metabolize the substrate for 24 h. Media was collected and extracted with and equal volume of 50/50 methanol/chloroform. Fluorescent metabolites extracted in the organic phase were collect and dried down before resuspension in 5 ul before spotting and running TLC as above.

In situ Ceramidase assay using C12-NBD-Ceramide. Cells (25,000/well) were plated in 96-well plates and grown overnight in the presence of +/−doxycycline (dox) (μg/ml) to turn on overexpression expression of dox inducible ceramidase. The next morning media was removed and replaced with 50 ul phenol red-free DMEM plus FBS and Pen/Strep media containing ceramidase inhibitor (in DMSO) to a final concentration of 0-50 uM (0.5% DMSO final concentration). Cells were incubated with ceramidase inhibitor for 1 h prior to adding 10 uL of 150 uM RBM14C12 in phenol red free media (25 μM final concentration) (ASAH1 and ASAH2) or RBM14C16 (ACER2 and ACER3) overexpression cells. Cells allow to grow and metabolize the substrate for 4 h. Media was removed into a clean plate and 15 μL of methanol was added to stop reaction. One-hundred of 2.5 mg/ml sodium periodate in 0.1 M Glycine/NaOH pH 10.6 buffer was added and incubated in the dark for 1 h before measuring fluorescence at excitation of 355 nm and emission at 460 nm as above. Alamar blue reagent was diluted in normal media (100 uL) and added to the cells to measure cell toxicity caused by the ceramidase inhibitor. Cell viability or toxicity cause by the incubation with ceramidase inhibitor was calculated as a percentage relative to cells containing vehicle control. Ceramidase activity (percent of activity remaining) was calculated first by subtracting the fluorescence reading of parental ASAH1 and ACER3 KO cells to subtract any remaining activity of these cells. Once the background reading was subtracted remaining activity was calculated as percent activity remaining versus inhibitor vehicle control.

Results and Discussion

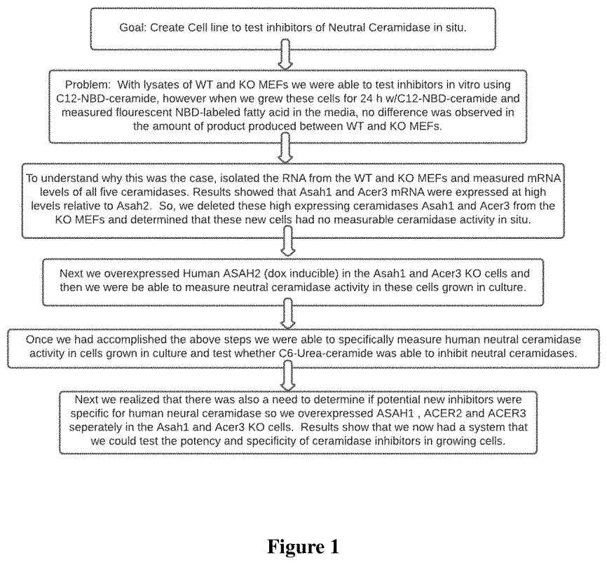

Aspects of the work performed are schematically outlined by the flow chart in FIG. 1.

This work began with efforts to develop an in situ/in cell assay that could be used to measure the effect of possible inhibitors of neutral ceramidase in mammalian cells. During this effort, we realized that to determine specificity of a possible inhibitors of neutral ceramidase, we would also need assays for the other ceramidases.

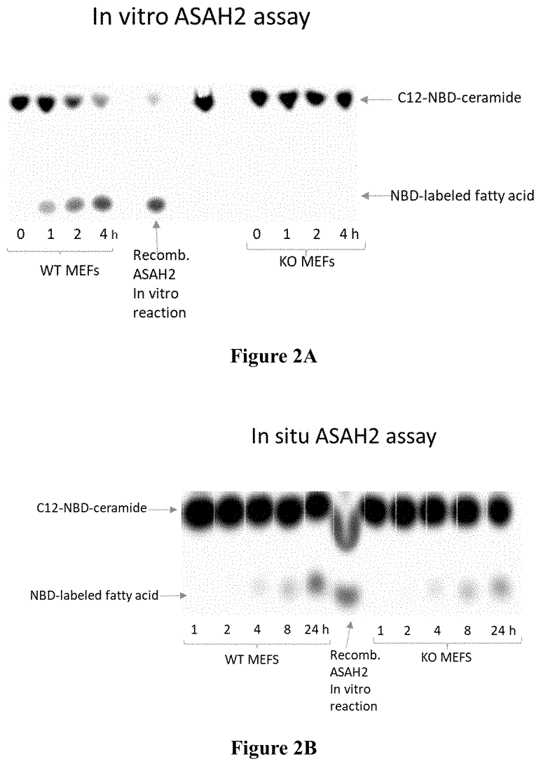

Initially, we began with two immortalized mouse embryonic fibroblast cell lines: one that had normal expression (WT) levels of ASAH2 and another cell line that was derived from a similar mouse that had ASAH2 functionally knocked out. In the in vitro assay, the reaction components (cell lysate (WT or KO MEFs as enzyme source), NBD-labeled C12-Ceramide (substrate) in a buffer of 50 mM HEPES, 150 mM sodium chloride and 1% sodium chloate, pH 7.2) were combined, and the substrate was converted to NBD labeled C12-shingosine by the ceramidase enzyme over time. Once the reaction was stopped the reaction components were separated using thin-layer chromatography (TLC). Doing assays of cell lysates on these cell lines demonstrated that the WT line has measurable ASAH2 activity, and as expected the KO cell line has no detectable levels of Asah2 activity in vitro (FIG. 2A).

We next wanted to see if the results from the in vitro assay using cell lysates would hold true if we incubated ASAH2 WT and KO MEFs grown in culture (in situ) with the same NBD-labeled C12-ceramide. Thus, we took an equal number of both ASAH2 WT and KO MEFS cells and plated them in cell culture dishes and allowed them to attach overnight. The next morning, we replace the media with media containing NBD-C12-ceramide (25 μM) and allowed the cells to grow and metabolize the NBD substrate. At various time after the addition of the substrate to the cells, we removed media and extracted the cells with an equal volume of chloroform/methanol (50/50). The mixture was centrifuged to separate two layers, and the lower organic layer was collected and evaporated to dryness. The resulting pellet was resuspended in 5 μL of chloroform/methanol (50/50) mix, loaded, and separated on a TLC plate. The results demonstrated that ASAH2 WT and KO MEFs produced equal amounts of the sphingosine product (FIG. 2B).

This result was initially unexpected. However, we soon arrived at an explanation. Mammalian cells include at least five ceramidase enzymes (1 acid ceramidase, 3 alkaline ceramidases, and at least 1 neutral ceramidase). The in vitro assay allows for pH control, and thus selectivity for certain ceramidases based on the pH of the reaction. However, during the in situ (in cell) assay, the NBD-C12-ceramide is merely added to the cell culture media. The substrate is subsequently transported into the cell to different organelles and metabolized there and then excreted from the cell. Neutral ceramidase is primarily located in the membranes of the cell, while acid ceramidase (aCDase) is located within lysosomes and alkaline ceramidases (ACERs 1, 2, and 3) are primarily located in the endoplasmic reticulum (ER) and Golgi complexes. As such, they can all possess different pH values and operate independently during the assay.

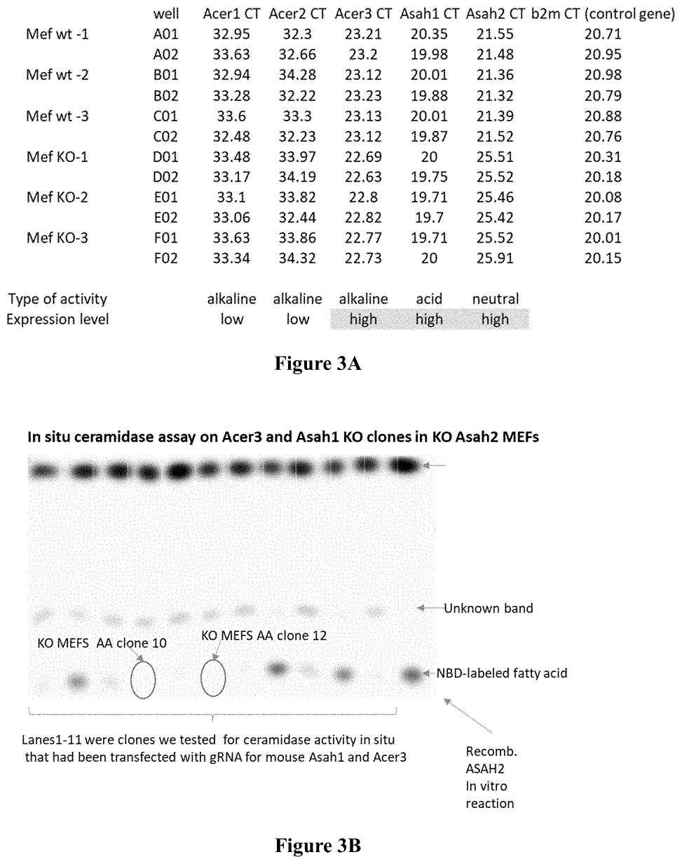

We next measured the mRNA levels of the various ceramidase in the ASAH2 WT and KO MEFs. After isolating RNA and making cDNA from the total RNA, we used real-time PCR to measure the relative abundance of the 5 different ceramidases in the Asah2 WT and KO MEFs (FIG. 3A). We determined that mRNA levels of ASAH1 and Acer3 as well as Asah2 were expressed at higher levels compared to the other ceramidases (FIG. 3A). Thus, we concluded that ASAH1 and ACER3 were likely the other ceramidase in the cell that were metabolizing the NBD-C12-ceramide in situ.

Next, we decided to see if we could knock out the function of ASAH1 and ACER3 in the ASAH2 KO MEFs using CRISPR-CAS9 technology. After infecting the cells with CAS9 and transfecting them with gRNAs targeting ASAH1 and ACER3, we isolated various clones and tested them for ceramidase activity in situ (FIG. 3B). Results showed through in situ enzyme assay and sequencing that we had successfully knocked out the function of ASAH1 and ACER3 in these cells, and we now had at least 2 different cell lines (KOAA10 and KOAA12) that had non-detectable levels of ceramidase activity (FIG. 3B).

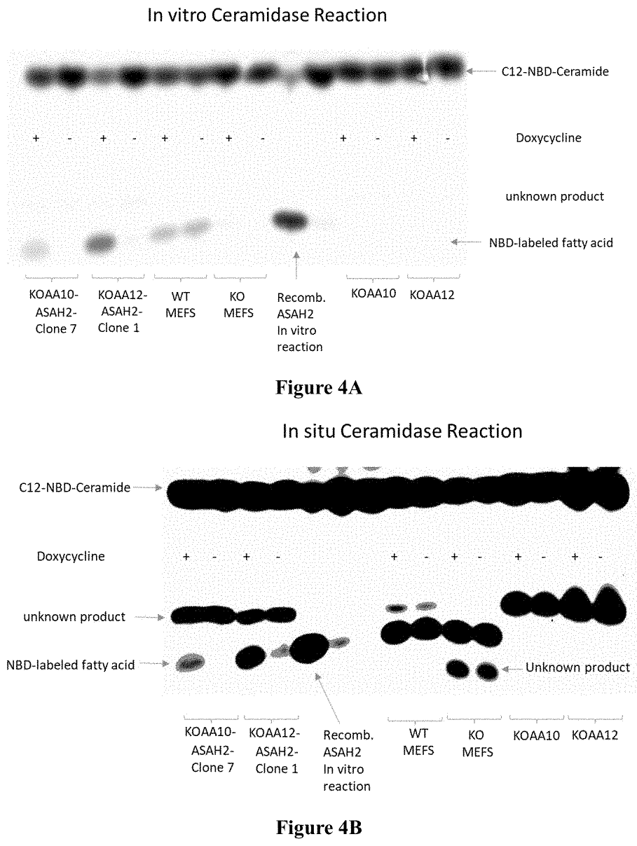

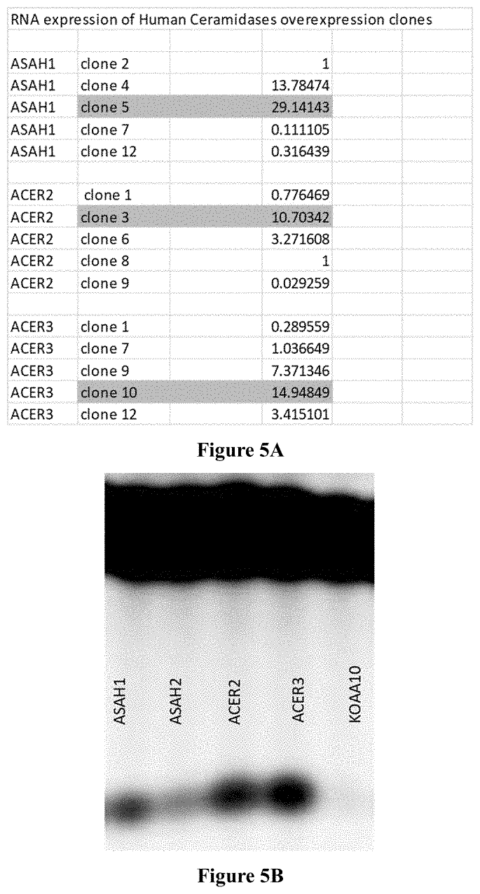

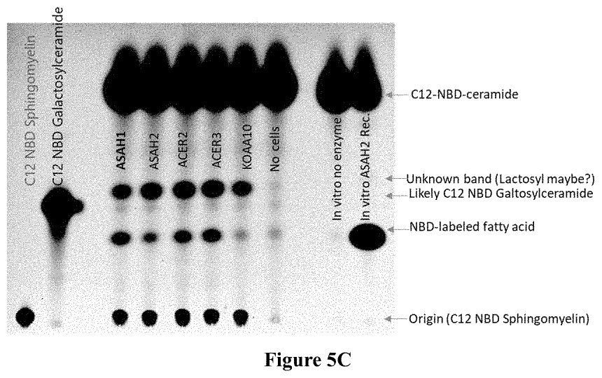

The next step was to overexpress the different ceramidase proteins separately in cell lines (KOAA10 and KOAA12) that had no detectable levels of ceramidase activity. ASAH2 was used initially for proof of principle studies (FIG. 4A-4B). Once this was successful, we used an analogous strategy with the other ceramidases including ASAH1, ACER2, and ACER3 from cDNA of mRNA into same doxycycline (DOX) inducible plasmid. After cloning the different ceramidase in the pCW57.1 doxycycline inducible vector separately, we confirmed that we had cloned the correct sequence into the plasmid. We used the different ceramidase plasmids to make a lentiviral virus (Addgene) that we used to infect the KOAA10 cells. After infection, antibiotic selection (puromycin) and isolation of individual clones we doxycycline induced these isolated clones to determine which clones had the highest levels of doxycycline inducible ceramidase activity. For ASAH1, ACER2 and ACER3 we used real-time rt-PCR to determine which clones had the highest mRNA expression (FIG. 5A) followed by in situ ceramidase assays to confirm that we had successful overexpression of the different ceramidase (FIGS. 5B-5C).

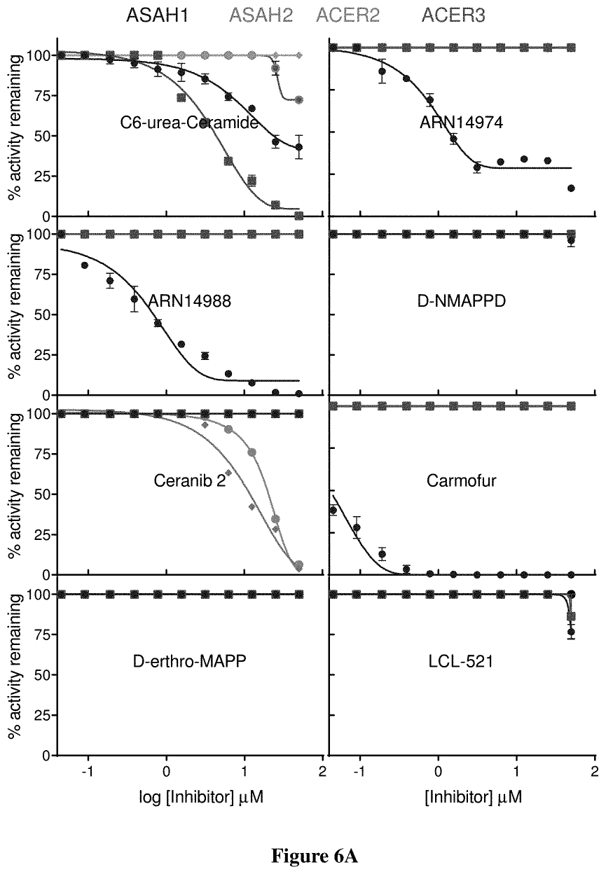

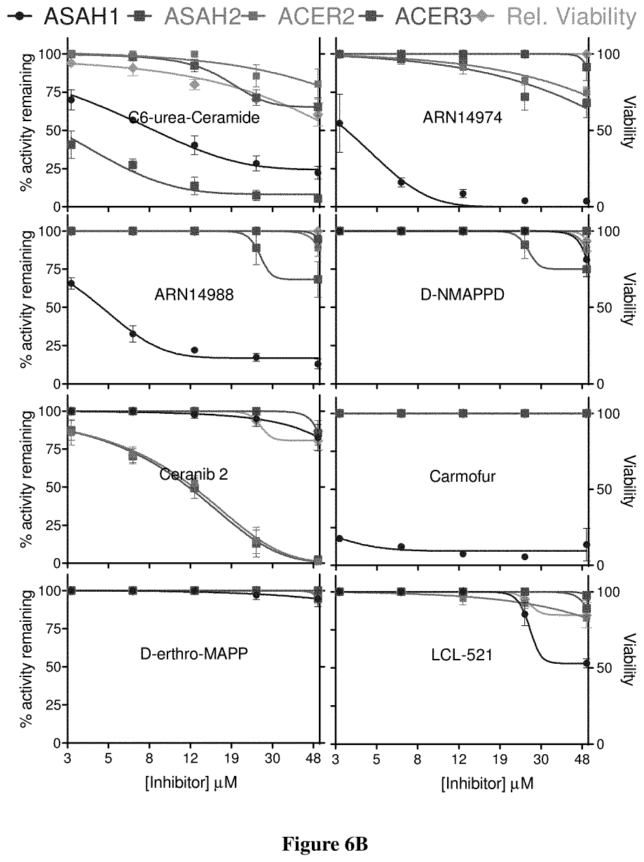

To validate our assay, we screened a panel of commercially available ceramidase inhibitors to determine if our cell lines and assays could be used to determine specificity and potency of the commercially available ceramidase inhibitors (FIGS. 6A-6B). As shown in FIGS. 6A-6B, with the parent cell line, using KOAA10 (for any background level of activity) along with the other ceramidase overexpression cell lines we can specifically test whether a potential inhibitor inhibits the correct ceramidase. Other in situ/in cell assays to test inhibitors of ceramidase have been developed; however, these assays have not included Acer2 in their specificity determination. Further, it is possible that other ceramidases are expressed in these cell lines that may complicate or confuse analysis of results. The improved assays described herein overcome these shortcomings.

The cell lines, kits, and methods of the appended claims are not limited in scope by the specific compounds, compositions, cell lines, kits, and methods described herein, which are intended as illustrations of a few aspects of the claims. Any cell lines, kits, and methods that are functionally equivalent are intended to fall within the scope of the claims. Various modifications of the cell lines, kits, and methods in addition to those shown and described herein are intended to fall within the scope of the appended claims. Further, while only certain representative compounds, components, compositions, cell lines, kits, reactions, and method steps disclosed herein are specifically described, other combinations of the compounds, components, compositions, cell lines, kits, reactions, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

1. A modified non-human cell deficient in the expression of one or more endogenous ceramidase genes and comprising one or more nucleic acids encoding a human ceramidase.

2. The modified non-human cell of claim 1, wherein the one or more endogenous ceramidase genes are selected from ASAH1, ASAH2, ASAH2B, ASAH2C, ACER1, ACER2, ACER3, or any combination thereof.

3. The modified non-human cell of claim 1, wherein the one or more endogenous ceramidase genes are selected from ASAH1, ASAH2, ACER1, ACER2, ACER3, or any combination thereof.

4. The modified non-human cell of claim 1, wherein the cell is deficient in the expression of ASAH1, ASAH2, ACER1, ACER2, and ACER3.

5. The modified non-human cell of claim 1, wherein the cell is deficient in the expression of all endogenous ceramidase genes.

6. The modified non-human cell of claim 1, wherein the cell exhibits no detectable ceramidase activity prior to introduction of the one or more nucleic acids encoding a human ceramidase.

7. The modified non-human cell of claim 1, wherein the one or more nucleic acids encode a human ceramidase selected from the group consisting of ASAH1, ASAH2, ASAH2B, ASAH2C, ACER1, ACER2, ACER3, or any combination thereof.

8. The modified non-human cell of claim 1, wherein the one or more nucleic acids encode a human ceramidase selected from the group consisting of ASAH1, ASAH2, ACER1, ACER2, ACER3, or any combination thereof.

9. The modified non-human cell of claim 1, wherein the one or more nucleic acids encode a single human ceramidase,
optionally wherein the single human ceramidase selected from the group consisting of ASAH1, ASAH2, ACER1, ACER2, ACER3, or any combination thereof.

10. The modified non-human cell of claim 1, wherein the cell is deficient in the expression of all endogenous ceramidase genes and comprises a nucleic acid encoding human ASAH1.

11. The modified non-human cell of claim 1, wherein the cell is deficient in the expression of all endogenous ceramidase genes and comprises a nucleic acid encoding human ASAH2.

12. The modified non-human cell of claim 1, wherein the cell is deficient in the expression of all endogenous ceramidase genes and comprises a nucleic acid encoding human ACER1.

13. The modified non-human cell of claim 1, wherein the cell is deficient in the expression of all endogenous ceramidase genes and comprises a nucleic acid encoding human ACER2.

14. The modified non-human cell of claim 1, wherein the cell is deficient in the expression of all endogenous ceramidase genes and comprises a nucleic acid encoding human ACER3.

15. The modified non-human cell of claim 1, wherein the reduced expression of the one or more endogenous ceramidase genes is obtained by deletion of all or part of the ceramidase gene, siRNA or oligonucleotide targeting the endogenous ceramidase gene, an inducible knockout system, or CRSIPR-CAS9.

16. A system for detecting the specificity and/or potency of a ceramidase inhibitor comprising the modified non-human cell of any of claim 1.

17. The system of claim 16, comprising a first modified non-human cell, a second modified non-human cell, a third modified non-human cell, a fourth modified non-human cell, and a fifth modified non-human cell,
wherein the first modified non-human cell is deficient in the expression of one or more endogenous ceramidase genes and comprises a nucleic acid encoding human ASAH1;
wherein the second modified non-human cell is deficient in the expression of one or more endogenous ceramidase genes and comprises a nucleic acid encoding human ASAH2;
wherein the third modified non-human cell is deficient in the expression of one or more endogenous ceramidase genes and comprises a nucleic acid encoding human ACER1;
wherein the fourth modified non-human cell is deficient in the expression of one or more endogenous ceramidase genes and comprises a nucleic acid encoding human ACER2; and
wherein the fifth modified non-human cell is deficient in the expression of one or more endogenous ceramidase genes and comprises a nucleic acid encoding human ACER3.

18. (canceled)

19. (canceled)

20. (canceled)

21. The system of claim 17,
wherein the first modified non-human cell is deficient in the expression of all endogenous ceramidase genes and comprises a nucleic acid encoding human ASAH1;
wherein the second modified non-human cell is deficient in the expression of all endogenous ceramidase genes and comprises a nucleic acid encoding human ASAH2;
wherein the third modified non-human cell is deficient in the expression of all endogenous ceramidase genes and comprises a nucleic acid encoding human ACER1;
wherein the fourth modified non-human cell is deficient in the expression of all endogenous ceramidase genes and comprises a nucleic acid encoding human ACER2; and
wherein the fifth modified non-human cell is deficient in the expression of all endogenous ceramidase genes and comprises a nucleic acid encoding human ACER3.

22. A kit for detecting the specificity and/or potency of a ceramidate inhibitor comprising the modified non-human cell of claim 1.

23. (canceled)

24. A method of detecting the specificity of a ceramidase inhibitor comprising obtaining a modified non-human cell deficient in the expression of one or more endogenous ceramidase genes and comprising one or more nucleic acids encoding a human ceramidase;
contacting said modified non-human cell with the ceramidase inhibitor; and
assaying the expression of the human ceramidase of each cell;
wherein reduced expression of a human ceramidase relative to a non-treated control indicates that the ceramidase inhibitor is specific for the ceramidase expressed in the cell, indicates the potency of the ceramidase inhibitor, or a combination thereof.

25-32. (canceled)
